(19) United States
(12) Patent Application Publication
(10) Pub. No.: US 2017/0189372 A1
(43) Pub. Date: Jul. 6, 2017

(54) USE OF SUBSTITUTED CHROMAN COMPOUNDS

(71) Applicant: INSTYTUT BIOLOGII MEDYCZNEJ, Lódz (PL)

(72) Inventors: Magdalena LUKASIAK, Parzeczew (PL); Jaroslaw DASTYCH, Lódz (PL)

(21) Appl. No.: 15/315,362

(22) PCT Filed: Jun. 1, 2015
(86) PCT No.: PCT/EP2015/062175

§ 371 Date: Nov. 30, 2016

(30) Foreign Application Priority Data
Jun. 2, 2014 (PL) P.408407

(51) Int. Cl. A61K 31/352; A61K 31/7034

(52) U.S. Cl. CPC A61K 31/352; A61K 31/7034

(57) ABSTRACT

A substituted chroman compound includes R^{1a} and R^{1b} as substituents selected from hydrogen and phenyl optionally substituted with 1-3 substituents. R^{2a} and R^{2b} are hydrogen atoms. R^{3a} and R^{3b} are C1-C4 alkyls, or represent an oxo group, or are C5-C8 alkylene substituents. R^{3a} and R^{3b} with their attached carbon atom form a ring. Nitrogen atoms replace two non-adjacent carbon atoms in the main chain to the C5-8 alkylene, which is optionally substituted by a thioxo group. n is the number of the substituents Rz. z is the sequence number of the substituent R^{z}. (R^{z}) is a substituent on the benzene ring attached to a non-nodal carbon atom selected from hydroxyl, halogen, C2-C6 alkenyl, C1-C4 alkoxy, C1-4 alkanoyloxy, heterocyclic C4-C7 alkyloxy having an oxygen atom in the ring and optionally substituted with 1-3 substituents. The compound is used as a tautomer, enantiomer, diastereomer, mixture of enantiomers and/or mixture of diastereomers.

### FIELD OF INVENTION

The invention is related to medicinal use of substituted chroman compounds.

### BACKGROUND OF INVENTION

Immunosuppressive drugs that inhibit activity of immune system are used in prevention of rejection of organ transplants and in therapy of multiple inflammatory diseases including asthma, multiple sclerosis or rheumatoid arthritis. Immunosuppressive drugs have however many side effects. Upon administration of those drugs immune system loses ability to efficiently resist infections. Other risks associated with immunosuppressive therapy include hyperglycemia, liver or kidney injury. Since available immunosuppressive drugs possess many drawbacks, there is still a need for new immunosuppressive compounds with less side effects and possibly oriented on specific molecular targets.

### SUMMARY OF INVENTION

The aim of the invention is to provide a new medicinal use of the substituted chroman compounds.

According to the invention, a substituted chroman compound of formula I

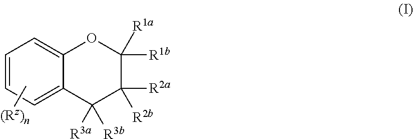

wherein
each of R^{1a} and R^{1b} is a substituent independently selected from the group consisting of hydrogen and phenyl optionally substituted with 1-3 substituents selected from the group consisting of halogen, hydroxyl, C1-C4 alkyl, C1-C4 alkoxy, C1-C4 alkanoyloxy,

R^{2a} and R^{2b} are hydrogen atoms,

each R^{3a} and R^{3b} is C1-C4 alkyl or R^{3a} and R^{3b} together represent an oxo group or together are an C5-C8 alkylene substituent so that together with the carbon atom to which R^{3a} and R^{3b} are attached form a ring, wherein in the main chain to C5-8 alkylene up to two non-adjacent carbon atoms are replaced by nitrogen atoms, and said C5-C8 alkylene is optionally substituted by a thioxo group,

n is the number of the substituents R^{z} and is an integer from 1 to 3 inclusive,

z is the sequence number of the substituent R^{z} and is an integer from 4 to n+3, inclusive,

(Rz) is a substituent on the benzene ring attached to a non-nodal carbon atom independently selected from the group consisting of hydroxyl, halogen, C2-C6 alkenyl, C1-C4 alkoxy, C1-4 alkanoyloxy, heterocyclic C4-C7 alkyloxy having an oxygen atom in the ring and optionally substituted with 1-3 substituents selected from the group consisting of C1-4 alkanoyloxy, phenyl-C3-6 alkenyl optionally substituted in the aryl ring by C1-4 alkanoyloxy and substituted in the chain by an oxo group located on a carbon atom adjacent to the carbon atom involved in the double bond C═C,

as tautomer, enantiomer, diastereomer, mixture of enantiomers and/or mixture of diastereomers,

is used as an immunosupressant drug.

In a preferred embodiment, the compound is a substituted chroman compound of formula I wherein:

each of R^{1a} and R^{1b} is a substituent selected from the group consisting of hydrogen and phenyl optionally substituted with 1-3 substituents selected from the group consisting of halogen, hydroxyl, methyl, methoxyl, acetoxyl,

each R^{3a} and R^{3b} is methyl or R^{3a} and R^{3b} together are an oxo group or together are a C5-C8 alkylene substituent so that together with the carbon atom to which R^{3a} and R^{3b} are attached form a ring, wherein in the main chain of C5-C8 alkylene two non-adjacent carbon atoms are replaced by nitrogen atoms, and said C5-C8 alkylene is optionally substituted by a thioxo,

(R^{z}) is a substituent on the benzene ring attached to a non-nodal carbon atom independently selected from the group consisting of hydroxyl, halogen, C3-C6 alkenyl, acetoxyl, tetrahydropyranyloxy and optionally substituted with 1-3 substituents selected from the group consisting of acetoxyl and phenyl-C4-C5 alkenyl optionally substituted in the aryl ring by acetoxyl and substituted in the chain by an oxo group located on a carbon atom adjacent to the carbon atom involved in the double bond C═C.

In particular, the substituted chroman compound is selected from the following:
6,8-dihydroxy-2-(4-methoxyphenyl)chroman-4-one
Spiro[4H-1-benzopyran-4,4′(1′H)-pyrimidine]-2′(3′H)-thione, 2,3,5′,6′-tetrahydro-7-hydroxy-6′,6′-dimethyl-2-phenyl (CAS: 299897-44-2),
4-(7-{6-[(3E)-4-(4-acetyloxyphenyl)-2-oxobut-3-enyl]-3,4,5-triacetyloxy(2H-3,4,5,6-tetrahydropyran-2-yloxy)}(2R)-5-acetyloxy-4-oxochroman-2-yl)phenyl acetate,
7-acetoksy-2,3,5′,6′-tetrahydro-6′,6′-dimetylo-2-fenylo-spiro[4H-1-benzopirano-4,4′(1′H)-pirymidyno]-2′(3′H)-tion (CAS: 299897-45-3),
5,7-dihydroxy-2-(4-hydroxyphenyl)-8-(3-methylbut-2-enyl)chroman-4-one
6-hydroxy-2-(2-hydroxyphenyl)chroman-4-one
2-(2,3-dimethoxyphenyl)-7-methoxychroman-4-one
5,7-dimethoxy-2-phenylchroman-4-one
5-methoxy-2-phenylchroman-4-one
2-(2,3-dimethoxyphenyl)chroman-4-one
7-methoxy-2-phenylchroman-4-one
2-(2,4-diacetyloxy-5-chlorophenyl)-6-chloro-2,4,4-trimethylchroman-7-yl acetate

### DETAILED DESCRIPTION

The activity of immunosuppressive drugs that are inter alia used in the transplantation of organs and in treatment of autoimmune diseases involves inhibition of the activity of immune system. There is a variety of immunosuppressive drugs employed in therapy, but still there is an ongoing search for a new substances having immunosuppressive properties.

Traditional in vivo models of immunosuppression are based mainly on solid organ grafts in rodents. There exist also methods based on rejection of tumor cells, e.g. rejection of Sal fibrosarcoma line in mice (Hammond-McKibben, D., P. Lake, et al. (2001); “A high-capacity quantitative mouse model of drug-mediated immunosuppression based on rejection of an allogeneic subcutaneous tumor”, J. Pharmacol. Exp. Ther., 297(3): 1144-51).

Widely used is a method of delayed type hypersensitive reaction in which mice is injected intraperitoneally with sheep red blood cells (SRBC) and receives the potential immunosuppressive agent. After several days animals are challenged by injecting SRBC intradermally in hind foot pad and the difference between footpad thickness between 0 and 24 h is measured (Akerkar, A. S., C. A. Ponkshe, et al. (2009); “Evaluation of immunomodulatory activity of extracts from marine animals”, Indian Journal of Marine Sciences, 38(1): 22-27). The throughput of such tests is higher but still does not match the capacity of HTS (high-throughput screening) methods which are an excellent alternative to in vivo tests in preliminary drug research. Those may be however less reliable as in vitro models do not detect the impact of potential drug on the whole immune system or all types of immune cells but concentrate generally on one cell type.

An example of using HTS methods in screening for immunosuppressive drugs was an assay based on mice dendritic cells that express YFP gene under the control of the IL-1β promoter designed to detect compounds inducing maturation of dendritic cells (Mizumoto, N., J. Gao, et al. (2005); “Discovery of novel immunostimulants by dendritic-cell-based functional screening”, Blood, 106(9): 3082-9). In this screening two chemical libraries consisting of 1986 and 880 compounds respectively were investigated and three compounds: camptothecine, colchicine and podophyllotoxin were identified as potential immunostimulants.

The inventors of the present invention in the course of performed screening research surprisingly found that certain compounds of substituted chroman selected from the vast group of compounds of different structures, said group very generally known as flavonoids exhibit immunosuppressive activity and therefore may be useful as active ingredients of immunosuppressive drugs and they can be used for the preparation of pharmaceutical formulations with immunosuppressive activity.

The present invention thus relates to a new medicinal use of a substituted chroman compound of formula I

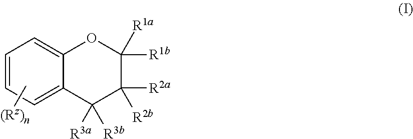

wherein
each of R^{1a} and R^{1b} is a substituent selected independently from the group consisting of hydrogen and phenyl optionally substituted with 1-3 substituents selected from the group consisting of halogen, hydroxyl, C1-C4 alkyl, C1-C4 alkoxy, C1-C4 alkanoyloxy,

R^{2a} and R^{2b} are hydrogen atoms

R^{3a} and R^{3b} is C1-C4 alkyl or R^{3a} and R^{3b} together represent an oxo group or together are an C5-C8 alkylene substituent so that together with the carbon atom to which R^{3a} and R^{3b} are attached form a ring, wherein in the main chain to C5-8 alkylene up to two non-adjacent carbon atoms are replaced by nitrogen atoms, and said C5-C8 alkylene is optionally substituted by a thioxo group,

n is the number of the substituents R^{z} and is an integer from 1 to 3 inclusive,

z is the sequence number of the substituent R^{z} and is an integer from 4 to n+3, inclusive,

(Rz) is a substituent on the benzene ring attached to a non-nodal carbon atom independently selected from the group consisting of hydroxyl, halogen, C2-C6 alkenyl, C1-C4 alkoxy, C1-4 alkanoyloxy, heterocyclic C4-C7 alkyloxy having an oxygen atom in the ring and optionally substituted with 1-3 substituents selected from the group consisting of C1-4 alkanoyloxy, phenyl-C3-6 alkenyl optionally substituted in the aryl ring by C1-4 alkanoyloxy and substituted in the chain by an oxo group located on a carbon atom adjacent to the carbon atom involved in the double bond C═C,

as tautomer, enantiomer, diastereomer, mixture of enantiomers and/or mixture of diastereomers.

In formula I, if either pair of the substituents R^{1a} and ^{1b} or R^{3a} and R^{3b} are substituents different from each other, the non-nodal ring carbon atoms may be asymmetrically substituted. The asymmetrically substituted carbon atoms may also exist in substituents of the chroman compound. Thus, the compounds of formula I can be optically active and exist as a mixture of enantiomers having different contents of individual enantiomers or in the form of essentially pure enantiomers. Moreover, as a result of the asymmetric substitution of more than one carbon atom, the compounds of formula I may exist both as a mixture of diastereomers with different contents of the individual diastereomers and substantially pure diastereomers.

Furthermore, compounds of formula I may exist as tautomers. Especially if R^{3a} and R^{3b} together represent oxo, a compound of formula I may exist in the tautomeric enol form.

In a preferred embodiment, the compound is a substituted chroman compound of formula I wherein:

each of R^{1a} and R^{1b} is a substituent selected from the group consisting of hydrogen and phenyl optionally substituted with 1-3 substituents selected from the group consisting of halogen, hydroxyl, methyl, methoxyl, acetoxyl,

each R^{3a} and R^{3b} is methyl or R^{3a} and R^{3b} together are an oxo group or together are a C5-C8 alkylene substituent so that together with the carbon atom to which R^{3a} and R^{3b} are attached form a ring, wherein in the main chain of C5-C8 alkylene two non-adjacent carbon atoms are replaced by nitrogen atoms, and said C5-C8 alkylene is optionally substituted by a thioxo,

(R^{z}) is a substituent on the benzene ring attached to a non-nodal carbon atom independently selected from the group consisting of hydroxyl, halogen, C3-C6 alkenyl, acetoxyl, tetrahydropyranyloxy and optionally substituted with 1-3 substituents selected from the group consisting of acetoxyl and phenyl-C4-C5 alkenyl optionally substituted in the aryl ring by acetoxyl and substituted in the chain by an oxo group located on a carbon atom adjacent to the carbon atom involved in the double bond C═C.

Especially preferably, the compound substituted chroman is selected from the following:
(1) 6,8-dihydroxy-2-(4-methoxyphenyl)chroman-4-one
(2) Spiro[4H-1-benzopyran-4,4′(1′H)-pyrimidine]-2′(3′H)-thione, 2,3,5′,6′-tetrahydro-7-hydroxy-6′,6′-dimethyl-2-phenyl (CAS: 299897-44-2),
(3) 4-(7-{6-[(3E)-4-(4-acetyloxyphenyl)-2-oxobut-3-enyl]-3,4,5-triacetyloxy(2H-3,4,5,6-tetrahydropyran-2-yloxy)}(2R)-5-acetyloxy-4-oxochroman-2-yl)phenyl acetate
(4) 7-acetoksy-2,3,5′,6′-tetrahydro-6′,6′-dimetylo-2-fenylo-spiro[4H-1-benzopirano-4,4′(1′H)-pirymidyno]-2′(3′H)-tion (CAS: 299897-45-3),
(5) 5,7-dihydroxy-2-(4-hydroxyphenyl)-8-(3-methylbut-2-enyl)chroman-4-one
(6) 6-hydroxy-2-(2-hydroxyphenyl)chroman-4-one
(7) 2-(2,3-dimethoxyphenyl)-7-methoxychroman-4-one
(8) 5,7-dimethoxy-2-phenylchroman-4-one
(9) 5-methoxy-2-phenylchroman-4-one
(10) 2-(2,3-dimethoxyphenyl)chroman-4-one
(11) 7-methoxy-2-phenylchroman-4-one
(12) 2-(2,4-diacetyloxy-5-chlorophenyl)-6-chloro-2,4,4-trimethylchroman-7-yl acetate

The inventors of the present invention employed cell based assay Fluorescent Cell Chip (FCC), which allows identification of substances that inhibit cytokine production in T cells. Screening were conducted on compounds from the chemical library of flavonoids.

### EXAMPLES

### Materials

All reagents were purchased from Sigma Chemical Co. (St. Louis, Mo., USA) unless stated otherwise. FBS was obtained from PAA Laboratories (Pasching, Austria), RPMI 1640 from Invitrogen Inc. (Carlsbad, Calif., USA) and Isoton for flow cytometry from Beckman Coulter (Brea, Calif., USA). The screening compound library was purchased from Prestwick Chemical (Illkirch, France) and was composed of 1120 marketed drugs.

### Cell Lines and Cell Culture

Development of reporter cell lines was described previously (Ulleras, E., D. Trzaska, et al. (2005); “Development of the “Cell Chip”: a new in vitro alternative technique for immunotoxicity testing”, Toxicology 206(2): 245-56). Briefly, EL-4 cells were stably transfected with transgene consisting of promoter regions from mouse β-actin, interleukin-2 (IL-2), interleukin-4 (IL-4), interferon-γ (IFN-γ), and interleukin-10 (IL-10) and ORF for EGFP. Reporter cells were cultured at density 5×10⁵-1×10⁶ cells/ml in a humidified atmosphere with 5% CO₂ at 37° C. in RPMI 1640 with Glutamax supplemented with 20% FCS and 0.5 μg/ml G418.

### High-Throughput Screening

When applicable, cells and reagents handling was performed by JANUS Automated Workstation (PerkinElmer, Waltham, Mass., USA). Cells were seeded at a density of 5×10⁴ per well in 96-well plates. In the primary screening reporter cells for IL-2 activated with PMA (50 ng/ml) and ionomycin (1 μM) were seeded in a 96-well plate and incubated with compounds from the chemical library in a total volume of 100 μL at 37° C. in a humidified atmosphere with 5% CO₂ for 18 h. Concentration of compounds was 0.4 μg/ml. The final DMSO concentration was 0.2%, DMSO is not cytotoxic towards EL-4 reporter cells at this concentration. Control samples consisted of cells in medium with 0.2% DMSO with PMA (50 ng/ml) and ionomycin (1 μM). Following incubation cells were analyzed by flow cytometry. Viability was assessed in parallel to the level of EGFP-mediated fluorescence.

To the next stage selected were compounds that most strongly inhibited EGFP fluorescence mediated by activation with and PMA and ionomycine and at the same time did not reveal high cytotoxicity in IL-2 reporter cell line. Selection of compounds was based on FCC parameters calculated using the following formula:

FCC parameter=relative viability×(relative fluorescence−100)

Compounds after primary screening were considered to be hits when FCC parameter calculated after primary screening was ≦−5000. After secondary screening area under the curve (A) of FCC parameter versus relative concentration was calculated (maximum concentration used, i.e. 50 μg/ml, was assumed to be 1).

Hit compounds were subjected to FCC test with 5 reporter cell lines. The test was conducted the same way as the primary screening but reporter cell lines for β-actin, interleukin-2, interleukin-4, interferon-γ, and interleukin-10 were used and the compounds were tested at the following concentrations: 0.05, 0.1, 0.2, 0.4, 0.8, 1.6 and 3.2 μg/ml. DMSO concentration was always 0.2%. The test was repeated 3 times.

### ELISA for IL-2

Jurkat cells were seeded in 48-well plates at a concentration of 1 million/ml. Cells activated with PMA (50 ng/ml) and ionomycin (1 uM) or resting were incubated with 50 selected flavonoids in a concentration of 10 μg/ml and 50 μg/ml for 24 hours in a humidified atmosphere with 5% CO 2 at 37° C. As a control, the respective cells in the medium or in a medium with PMA and ionomycin were employed. After incubation, cells were centrifuged (8 minutes, 1300 rpm), and the cell-free supernatants were stored at −20° C. until measurements. IL-2 was assessed using a commercially available ELISA kits according to the manufacturer's instructions. Each experiment was repeated 3 times.

### Data Validation and Statistical Analysis

FCC assay and ELISA assay were repeated three times. Relative data were presented as the means±SEM. Test FCC oraz test ELISA zosta [symbol] powtórzony 3 razy. Wzgl [symbol] dne dane przedstawiono jako średnie ±SEM. Statistical significance of differences observed in both assays was tested with ANOVA followed by post hoc Dunnett test using GraphPad software (La Jolla, Calif., USA). In statistical analysis non-relative data were used

### FCC Test

Flavonoids selected after preliminary screening were undergoing FCC testing using 5 reporter cell lines: for β-actin, interleukin-2 (IL-2), interleukin-4 (IL-4), interferon-γ (IFN-γ) and interleukin-10 (IL-10). The tested concentrations were in the range from 1.56 to 50 μg/ml. The results are shown in Table 1.

TABLE 1
The value of the FCC parameter in the screening with reporter cell line for IL-2;
value of the area under the curve showing the relationship between the FCC
parameter and compound concentration in the expanded version of FCC assay;
The percent of inhibition of IL-2 production (ELISA)

| | | The area under the curve showing | | | | | | |
|---|---|---|---|---|---|---|---|---|
| | FCC | the relationship between the FCC | | | | | Inhibition of IL-2 | |
| | parameter | parameter and compound | | | | | production (ELISA) | |
| Compound | (IL-2 | concentration | | | | | [%] | |
| no. | screening) | AKT | IL-2 | IL-4 | IFN | IL-10 | 10 μg/ml | 50 μg/ml |
| 1 | −8723 | −622 | −2178 | −194 | −1865 | −1435 | No | 97 |
| | | | | | | | inhibition | |
| 2 | −5484 | −1120 | −2394 | 261 | −3977 | −2585 | 35 | 91 |
| 3 | −5095 | 945 | −4581 | −308 | −3801 | −1509 | 64 | 68 |
| 4 | −5692 | −104 | −1081 | 988 | −3521 | −1928 | 27 | 44 |
| 5 | −5264 | −979 | −4991 | −3324 | −4565 | −3119 | 28 | 95 |
| 6 | −5169 | 3622 | −2227 | 367 | −2999 | −179 | 58 | 100 |
| 7 | −6178 | −1357 | −4104 | −1865 | −5873 | −2889 | 67 | 92 |
| 8 | −5788 | 3973 | −2590 | 2380 | −4396 | −628 | 80 | 100 |
| 9 | −5636 | 3861 | −3872 | 675 | −4745 | −1472 | 43 | 92 |
| 10 | −6685 | 118 | −2244 | −946 | −3142 | −1336 | 32 | 97 |
| 11 | −6917 | −754 | −3670 | −1499 | −3975 | −777 | 35 | 97 |
| 12 | −7615 | −2936 | −6892 | −5349 | −5259 | −2749 | 100 | 83 |

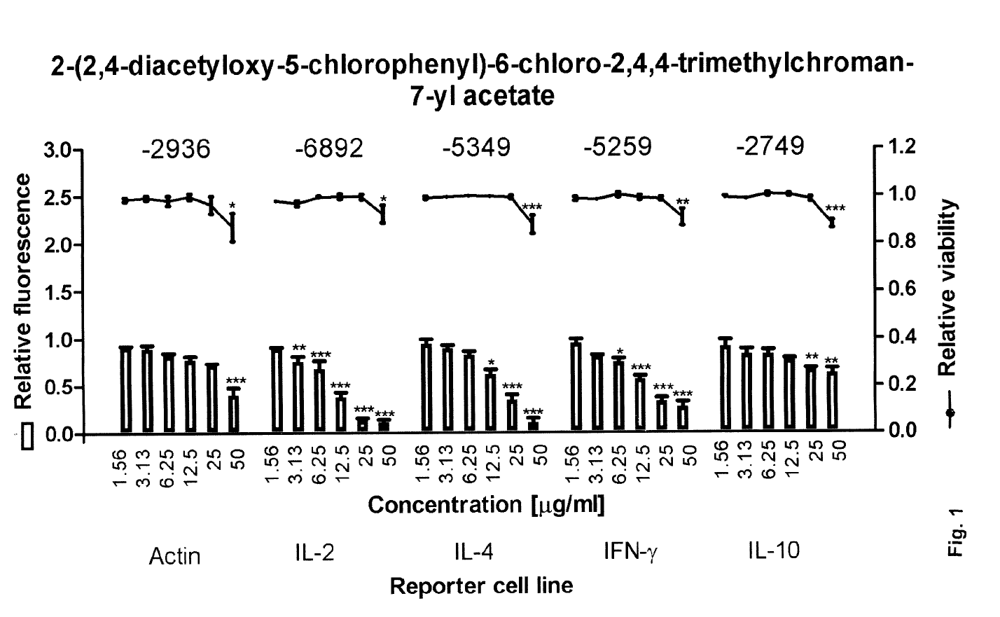

Most of the substituted chroman compounds according to the invention strongly inhibit PMA and ionomycin induced EGFP-mediated fluorescence as indicated by the value of area under the curve showing the relationship between FCC parameter and the concentration. Results of the FCC test obtained for substituted chroman compound No. (12), in which the highest inhibition of PMA and ionomycin induced fluorescence of EGFP was observed and which at the same time strongly inhibited PMA-induced and ionomycin IL-2 production by ELISA are shown in FIG. 1.

FIG. 1 illustrates the relative fluorescence and relative viability of reporter cells in the assay FCC for the compound No. (12)-2-(2,4-diacetyloxy-5-chlorophenyl)-6-chloro-2,4,4-trimethylchroman-7-yl acetate

Reporter cell lines for β-actin, IL-2, IL-4, IFN-γ and IL-10 activated with PMA and ionomycin were incubated with serial dilutions of the compound No. (12).

Using flow cytometry mean fluorescence intensity (columns, left Y axis) and cell viability (dots, the right Y-axis) was determined for all the experimental points and normalized relative to the control—cells incubated with medium with PMA and ionomycin. The data is the mean of the three experiments ±SEM. The statistical significance of differences between means were determined: * p<0.05, ** p<0.01, *** p<0.001 versus control. Values above the graph represent the area under the curve showing the relationship of the FCC parameter and the relative compound concentration.

Compound No. (12) according to the invention caused a statistically significant decrease in EGFP-mediated fluorescence in the case of the IL-2, IL-4 and IFN-γ reporter cell lines (area below −5000), and to a lesser extent, the reporter cell line for IL-10 (the area between the −2000 and −3000). Compound No. (12) was not cytotoxic in the reporter cell line EL-4 at concentrations up to 25 mg/ml, even that at concentration of 25 μg/ml resulted in a decrease in EGFP-mediated fluorescence in reporter cell lines for IL-2 and IL 4 by more than 90%.

Compounds No. (1)-(10) according to the invention inhibited PMA and ionomycin induced IL-2 production, at least at a concentration of 50 μg/ml.

1. Substituted chroman compound of formula I: 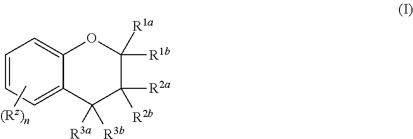
wherein
each of R^{1a} and R^{1b} is a substituent independently selected from the group consisting of hydrogen and phenyl optionally substituted with 1-3 substituents selected from the group consisting of halogen, hydroxyl, C1-C4 alkyl, C1-C4 alkoxy, C1-C4 alkanoyloxy,
R^{2a} and R^{2b} are hydrogen atoms
each R^{3a} and R^{3b} is C1-C4 alkyl or R^{3a} and R^{3b} together represent an oxo group or together are an C5-C8 alkylene substituent so that together with the carbon atom to which R^{3a} and R^{3b} are attached form a ring, wherein in the main chain to C5-8 alkylene up to two non-adjacent carbon atoms are replaced by nitrogen atoms, and said C5-C8 alkylene is optionally substituted by a thioxo group,
n is the number of the substituents R^{z} and is an integer from 1 to 3 inclusive,
z is the sequence number of the substituent R^{z} and is an integer from 4 to n+3, inclusive,
(R^{z}) is a substituent on the benzene ring attached to a non-nodal carbon atom independently selected from the group consisting of hydroxyl, halogen, C2-C6 alkenyl, C1-C4 alkoxy, C1-4 alkanoyloxy, heterocyclic C4-C7 alkyloxy having an oxygen atom in the ring and optionally substituted with 1-3 substituents selected from the group consisting of C1-4 alkanoyloxy, phenyl-C3-6 alkenyl optionally substituted in the aryl ring by C1-4 alkanoyloxy and substituted in the chain by an oxo group located on a carbon atom adjacent to the carbon atom involved in the double bond C═C,
as tautomer, enantiomer, diastereomer, mixture of enantiomers and/or mixture of diastereomers.
for use as an immunosuppressant drug.

2. A compound according to claim 1 wherein:
each of R^{1a} and R^{1b} is a substituent selected from the group consisting of hydrogen and phenyl optionally substituted with 1-3 substituents selected from the group consisting of halogen, hydroxyl, methyl, methoxyl, acetoxyl,
each R^{3a} and R^{3b} is methyl or R^{3a} and R^{3b} together are an oxo group or together are a C5-C8 alkylene substituent so that together with the carbon atom to which R^{3a} and R^{3b} are attached form a ring, wherein in the main chain of C5-C8 alkylene two non-adjacent carbon atoms are replaced by nitrogen atoms, and said C5-C8 alkylene is optionally substituted by a thioxo,
(R^{z}) is a substituent on the benzene ring attached to a non-nodal carbon atom independently selected from the group consisting of hydroxyl, halogen, C3-C6 alkenyl, acetoxyl, tetrahydropyranyloxy and optionally substituted with 1-3 substituents selected from the group consisting of acetoxyl and phenyl-C4-C5 alkenyl optionally substituted in the aryl ring by acetoxyl and substituted in the chain by an oxo group located on a carbon atom adjacent to the carbon atom involved in the double bond C═C.

3. A compound according to claim 2, wherein that compound is selected from the following:
6,8-dihydroxy-2-(4-methoxyphenyl)chroman-4-one,
Spiro[4H-1-benzopyran-4,4′(1′H)-pyrimidine]-2′(3′H)-thione, 2,3,5′,6′-tetrahydro-7-hydroxy-6′,6′-dimethyl-2-phenyl (CAS: 299897-44-2),
4-(7-{6-[(3E)-4-(4-acetyloxyphenyl)-2-oxobut-3-enyl]-3,4,5-triacetyloxy(2H-3,4,5,6-tetrahydropyran-2-yloxy)}(2R)-5-acetyloxy-4-oxochroman-2-yl)phenyl acetate,
7-acetoksy-2,3,5′,6′-tetrahydro-6′,6′-dimetylo-2-fenylo-spiro[4H-1-benzopirano-4,4′(1′H)-pirymidyno]-2′(3′H)-tion (CAS: 299897-45-3),
5,7-dihydroxy-2-(4-hydroxyphenyl)-8-(3-methylbut-2-enyl)chroman-4-one,
6-hydroxy-2-(2-hydroxyphenyl)chroman-4-one,
2-(2,3-dimethoxyphenyl)-7-methoxychroman-4-one,
5,7-dimethoxy-2-phenylchroman-4-one,
5-methoxy-2-phenylchroman-4-one,
2-(2,3-dimethoxyphenyl)chroman-4-one,
7-methoxy-2-phenylchroman-4-one,
2-(2,4-diacetyloxy-5-chlorophenyl)-6-chloro-2,4,4-trimethylchroman-7-yl acetate.

4. Use of a substituted chroman compound of formula I according to claim. 1, for preparation of an immunosuppressant drug.

5. Use of a substituted chroman compound according to claim 4, wherein said compound is of formula I, wherein:
each of R^{1a} and R^{1b} is a substituent selected from the group consisting of hydrogen and phenyl optionally substituted with 1-3 substituents selected from the group consisting of halogen, hydroxyl, methyl, methoxyl, acetoxyl,
each R^{3a} and R^{3b} is methyl or R^{3a} and R^{3b} together are an oxo group or together are a C5-C8 alkylene substituent so that together with the carbon atom to which R^{3a} and R^{3b} are attached form a ring, wherein in the main chain of C5-C8 alkylene two non-adjacent carbon atoms are replaced by nitrogen atoms, and said C5-C8 alkylene is optionally substituted by a thioxo,
(R^{z}) is a substituent on the benzene ring attached to a non-nodal carbon atom independently selected from the group consisting of hydroxyl, halogen, C3-C6 alkenyl, acetoxyl, tetrahydropyranyloxy and optionally substituted with 1-3 substituents selected from the group consisting of acetoxyl and phenyl-C4-C5 alkenyl optionally substituted in the aryl ring by acetoxyl and substituted in the chain by an oxo group located on a carbon atom adjacent to the carbon atom involved in the double bond C═C.

6. Use of a substituted chroman compound according to claim 5, wherein said compound is selected from:
6,8-dihydroxy-2-(4-methoxyphenyl)chroman-4-one,
Spiro[4H-1-benzopyran-4,4′(1′H)-pyrimidine]-2′(3′H)-thione, 2,3,5′,6′-tetrahydro-7-hydroxy-6′,6′-dimethyl-2-phenyl (CAS: 299897-44-2),
4-(7-{6-[(3E)-4-(4-acetyloxyphenyl)-2-oxobut-3-enyl]-3,4,5-triacetyloxy(2H-3,4,5,6-tetrahydropyran-2-yloxy)}(2R)-5-acetyloxy-4-oxochroman-2-yl)phenyl acetate,
7-acetoksy-2,3,5′,6′-tetrahydro-6′,6′-dimetylo-2-fenylo-spiro[4H-1-benzopirano-4,4′(1′H)-pirymidyno]-2′(3′H)-tion (CAS: 299897-45-3),
5,7-dihydroxy-2-(4-hydroxyphenyl)-8-(3-methylbut-2-enyl)chroman-4-one,
6-hydroxy-2-(2-hydroxyphenyl)chroman-4-one,
2-(2,3-dimethoxyphenyl)-7-methoxychroman-4-one,
5,7-dimethoxy-2-phenylchroman-4-one,
5-methoxy-2-phenylchroman-4-one,
2-(2,3-dimethoxyphenyl)chroman-4-one,
7-methoxy-2-phenylchroman-4-one,
2-(2,4-diacetyloxy-5-chlorophenyl)-6-chloro-2,4,4-trimethylchroman-7-yl acetate.
